(19) United States
(12) Patent Application Publication
(10) Pub. No.: US 2024/0024227 A1
(43) Pub. Date: Jan. 25, 2024

(54) CANNABIS PLANT DERIVED EXTRACELLULAR VESICLES AND THERAPEUTIC METHODS USING THE SAME

(71) Applicant: UNIVERSITY OF FLORIDA RESEARCH FOUNDATION, INCORPORATED, Gainesville, FL (US)

(72) Inventors: Hassan AZARI, Gainesville, FL (US); Thomas D. SCHMITTGEN, Gainesville, FL (US); Nasser NASSIRI KOOPAEI, Alachua, FL (US); Brent REYNOLDS, Gainesville, FL (US)

(21) Appl. No.: 18/028,937

(22) PCT Filed: Sep. 28, 2021
(86) PCT No.: PCT/US2021/052284

§ 371 Date: Mar. 28, 2023

Related U.S. Application Data
(60) Provisional application No. 63/084,193, filed on Sep. 28, 2020.

(51) Int. Cl. A61K 9/00; A61K 31/00; A61P 35/00; A61P 25/28; A61K 36/185; A61K 9/51; A61M 15/08

(52) U.S. Cl. CPC A61K 9/0043; A61K 31/658; A61P 35/00; A61P 25/28; A61K 36/185; A61K 9/5176; A61M 15/08

(57) ABSTRACT

Disclosed is a method and composition for using plant extracellular vesicles (EVs) as vectors for delivering therapeutic molecules to the CNS. We demonstrated that plant EVs are more efficient vectors than delivery with other methods. These vectors can used to treat diseases in the CNS, such as cancer, injuries like TBI, degeneration such as Alzheimer's and aging and cognitive disorders such as PTSD, depression and anxiety. New data using hemp derived EVs to treat cancers in the brain and to impact precursor cells in the brain and isolation of EVs are included. In addition, disclosed is an improvement on the efficacy of intra-nasal delivery and oral delivery when using EVs.

### BACKGROUND

Cannabinoids have long been used for their remedy of different ailments such as pain, seizures, cancer chemotherapy associated symptoms such as nausea and vomiting, and also for their direct anti-cancer effects by inducing cell death in tumor cells. The main cannabinoids that have been used extensively are delta 9-tetrahydroxicannabinol (THC) and cannabidiol (CBD). These cannabinoids are extracted, purified and decarboxylated from different cannabis plant cultivars containing different cannabinoids at different ratios in raw acidic forms. While a lot of studies have been performed using decarboxylated cannabinoids, the scientific community has begun to appreciate therapeutic effects of cannabinoids in their acidic form such as the anti-tumor activity of cannabidiolic acid (CBD-A) and neuroprotective effects of tetrahydroxicannabinolic acid (THC-A).

Cannabinoids not only are limited in therapeutic applications by the types of cannabinoids being used but also by their delivery methods; oral, oro-mucusal, transdermal and intratumoral administration, each with its unique advantages and disadvantages. For therapy purpose in the central nervous system, in particular for brain tumors, to reach therapeutic concentrations (in brain tumor bed), patients are required to consume relatively high doses of cannabinoids orally on a daily basis. This is challenging for several reasons: 1—If cannabinoids are taken orally, they undergo extensive hepatic first-pass metabolism which reduces their plasma concentration and increases the psychoactive metabolites, 11-hydroxy THC, when THC is administered. 2—Oral administration results in a systemic distribution in the body and in the long term, their accumulation in well-vascularized and fatty organs due to the lipophilic property of cannabinoids. 3—systemic distribution and accumulation have adverse effects on cardiovascular, renal and hepatic function particularly in elderly people with background diseases. Moreover, administration of low doses to avoid psychoactive effects of THC may result in a suboptimal concentration in the tumor bed that may increase tumor proliferation. To avoid these problems in CNS tumor therapy, cannabinoids may be delivered intratumorally, which is invasive and traumatic. In addition, despite delivering a high concentration of the cannabinoids to the tumor core in the latter approach, an adequate amount may not reach tumor locations far from the infusion site, which in turn may result in poor therapeutic outcomes.

### BRIEF DESCRIPTION OF THE DRAWINGS

The present embodiments are illustrated by way of example, and not by way of limitation, in the figures of the accompanying drawings.

The following figures are illustrative only, and are not intended to be limiting

FIG. 1 shows hemp EVs characterization: A) EVs size distribution as determined by NTA. B) TEM picture of hemp EVs. C) Major cannabinoid content of hemp EVs based on LC-MS/MS analysis.

FIG. 2 shows tumor cells (Human GBM LO) staining with free SYTO™ RNASelect™ dye (A) versus uptaking SYTO™ RNASelect™ stained hemp EVs (B) after 3 hours of incubation.

FIG. 3 shows In vitro anti-proliferative [at different concentrations, 7 days] effects of hemp EVs on A) mouse KR158 vs (B) human LO GBM lines and (C) and anti-migration [at 111M for 24 hours] effects of hemp NPs on human LO GBM line in culture. *P<0.05, **P=0.001, ****P<0.0001.

FIG. 4 shows intranasal delivery of SYTO™ RNASelect™ stained hemp NPs into a mouse brain. 1 & 2 are respectively showing hemp NPs distribution in tumor [1] vs non-tumor [2] areas of a coronal brain section.

FIG. 5 shows microglial cell Iba-limmunostaining in control (A) vs hemp NPs treated (B) mouse brain. Hemp NP treatment changes the morphology and density of microglial cells in the brain.

FIG. 6 shows the effect of hemp NPs treatment on survival pf KR158 glioma-bearing mice. Hemp NPs treatment increased median survival (Log-rank test, **P=0.0016, 31 days [control] vs 42 days [Hemp NPs]).

FIG. 7 shows that hemp NPs treatment leads to accumulation of substantial amount of CBD-A and CBG-A in brain tissue.

FIG. 8 shows that hemp NPs treatment leads to accumulation of substantial amount of CBD-A and CBG-A in brain tumor tissue.

FIG. 9 shows that plant derived extracellular vesicles [EVs] improve intranasal delivery by 5-fold.

FIG. 10 shows that plant derived extracellular vesicles [EVs] improves oral delivery of drugs to the CNS.

FIG. 11 shows how plant derived EVs reduce inflammation in a TBI model.

FIG. 12 shows that IBA-1 expression levels are reduced in EV treated animals following TBI.

FIG. 13 shows how plant EVs target the Glioma progenitor and glioma stem cells.

FIG. 14 shows that hemp EVs differentially modulate Neural Stem Cell [NSC] and progenitor cell proliferation.

### DETAILED DESCRIPTION

While modulating the total amount of each constituent cannabinoid such as THC in drug formulations can improve on the breadth and effectiveness of these anti-glioma compounds, nano-packaging of cannabinoids, using acidic cannabinoids and employing targeted, local and noninvasive CNS delivery methods such as intranasal route are attractive translational approaches for cancers and other diseases of the CNS. These strategies reduce the overall administered dose of cannabinoids and their undesirable side effects, while maintaining or even enhancing cannabinoids effects on CNS disorders and anti-tumor efficacy.

In one embodiment, there is provided a method for treating brain cancer in a subject comprising administering plant extracellular vesicles (PEVs) containing one or more cannabinoids (CPEVs) to the subject wherein the CPEVs are delivered to the brain of the subject.

For example, methods described herein may be used for the treatment of glioblastoma. In certain aspects, the brain cancer is treated by administration of the PEVs intranasally, or alternatively parenterally. Delivery of the CPEVs to the brain can result in brain concentrations that are higher than serum concentrations resulting from other modes of administration (e.g. intravascular or oral administration) thereby allowing a dosage of cannabinoid containing CPEVs to be used which is effective to treat the brain cancer while not causing the aforementioned side effects commonly associated with cannabinoid administration in the subject.

In a second embodiment, provided is a method for treating neurodegenerative diseases by administering a therapeutically effective amount of CPEVs to the central nervous system of a subject. Examples of neurodegenerative diseases of the CNS treatable with the compositions described herein include, but are not limited to one or more of Alzheimer's disease, Parkinson's disease, Huntington's disease, motor neuron disease, spinocerebellar ataxia, and spinal muscular atrophy.

In a third embodiment, provided is a method for treat CNS injuries in a subject by administering a therapeutically effective amount CPEVs as described herein. Examples of CNS injuries by the methods disclosed herein include, but are not limited to as stroke, traumatic brain injury, concussion, spinal cord injury and the like.

In a fourth embodiment, provided is a method for reducing inflammatory conditions in the CNS of a subject by administering a therapeutically effective amount of CPEVs disclosed herein. Examples of inflammatory conditions treatable by the compositions described herein include auto-immune diseases (e.g. multiple sclerosis, encephalitis and the like).

In a fifth embodiment, provided is a method for augmenting function of the CNS by administering an effective amount of CPEVs as disclosed herein. Augmenting function of the CNS includes, but is not limited to reducing aging of the brain, maintaining or enhancing cognitive function, motor function and/or resilience to injury and disease.

In a sixth embodiment, provided is a method for enhancing the oral delivery of molecules to the CNS using a therapeutically effective amount of CPEV. This delivery method can be used to treat a variety of CNS diseases, dysfunctions, reduce inflammation and augment CNS function.

In some aspects, methods disclosed herein concern administration of composition comprising CPEVs to a subject such that the CPEVs are delivered to the brain of the subject. For instance, CPEVs can be administered intranasally or intracranially and, in various aspects, is administered 1, 2, 3, 4, 5 or more times. Intracranial administration of CPEVs is accomplished, in some aspects, by providing the CPEVs though a cannula. In certain other aspects, CPEVs provided in a liquid composition formulated for intranasal administration that typically includes an excipient for intranasal administration. Moreover, in some cases, intranasal administration of CPEVs is accomplished by applying pressure to an CPEV composition. Thus, in some cases, an CPEV composition for intranasal delivery is comprised in a syringe, a nebulizer, a respirator or a squeeze bottle such that pressure can be applied to facilitate CPEV delivery to the intranasal passages of the subject (e.g., via a mechanical action by a human or pump or via a compressed gas).

CPEVs may be provided in a variety of formulations any of which may be used for methods disclosed herein. In some cases, CPEV composition is formulated to enhance uptake to the brain from the intranasal passages.

In still further aspects, pharmaceutical compositions comprising CPEVs are provided. For example, in one aspect, the disclosure provides an CPEV composition for intracranial administration comprising CPEVs formulated in artificial cerebrospinal fluid (ACSF). Formulations for ACSF are known in the art and certain specific formulations are detailed herein. Alternatively, there is provided, a pharmaceutical composition for intranasal administration comprising a dosage of CPEV effective to treat brain cancer (e.g. glioblastoma) when administered to a subject via the intranasal route in a carrier formulated for intranasal administration. Moreover, in certain aspects, CPEV compositions are administered by a syringe, a nebulizer, a respirator (or a cartridge that is a designed for coupling to a nebulizer or respirator) or a squeeze bottle to facilitate intranasal administration. In still further aspects, CPEV compositions for intracranial or intranasal administration can comprise a second therapeutic agent such as, for example, a chemotherapeutic agent, an antiviral, an antibiotic or an anti-inflammatory agent. Other therapeutic agents may be added to the PEV composition by exogenous delivery, using known methods, or by genetically modifying the plant material with the PEVs are isolated from.

In yet further embodiments, the disclosure provides an intranasal CPEV delivery system comprising CPEVs formulated for intranasal delivery and a pressure source sufficient to deliver the CPEV to the intranasal passages of a subject. In certain aspects, the pressure source is defined as supplying sufficient pressure to deliver an CPEV composition to the intranasal passages of a subject. For example, the pressure source may be a syringe, a nebulizer, a respirator a squeeze bottle or a pump. In certain aspects, a CPEV delivery system comprises a single unit dosage of CPEV effective for addressing cancer, neurodegenerative diseases, inflammatory conditions, CNS injuries, or augmenting CNS function in a human subject. Thus, in still further aspects, there is provided a kit for the treatment of brain cancer, comprising one or more unit doses of CPEVs formulated for intranasal administration.

In additional embodiments, the disclosure provides uses of compositions described herein for the preparation of medicaments. For example, PEVs may be isolated from a plant such as hemp or other cannabis species, and engineered to include an exogenous payload. In such example, the PEVs are used a delivery vehicle for certain constituents and medicaments. Other related aspects are also provided in the instant invention.

The foregoing summary is not intended to define every aspect of the invention, and additional aspects are described in other sections, such as the following detailed description. The entire document is intended to be related as a unified disclosure, and it should be understood that all combinations of features described herein are contemplated, even if the combination of features are not found together in the same sentence, or paragraph, or section of this document. Other features and advantages of the invention will become apparent from the following detailed description. It should be understood, however, that the detailed description and the specific examples, while indicating preferred embodiments of the invention, are given by way of illustration only, because various changes and modifications within the spirit and scope of the invention will become apparent to those skilled in the art from this detailed description.

### Definitions

Where the definition of terms departs from the commonly used meaning of the term, applicant intends to utilize the definitions provided below, unless specifically indicated.

The terminology used herein is for the purpose of describing particular embodiments only and is not intended to be limiting. As used herein, the singular forms “a,” “an,” and “the” are intended to include the plural forms as well, unless the context clearly indicates otherwise these terms do not denote a limitation of quantity, but rather denote the presence of at least one of the referenced item. Furthermore, to the extent that the terms “including,” “includes,” “having,” “has,” “with,” or variants thereof are used in either the detailed description and/or the claims, such terms are intended to be inclusive in a manner similar to the term “comprising.”

The term “about” or “approximately” is meant to denote up to a 5, 6, 7, 8, 9, or 10 percent variance in the stated value or range. For example, about 2 includes values of 1.9 to 2.1.

As used herein, the term “adjunct cancer therapy protocol” refers to a therapy, such as surgery, chemotherapy, radiotherapy, thermotherapy, and laser therapy, and may provide a beneficial effect when administered in conjunction with administration

As used herein, the term “an amount” refers to a statistically significant amount.

The term “cancer” as used herein means is intended to include any neoplastic growth in a patient, including an initial tumor and any metastases. The cancer can be of the liquid or solid tumor type. Liquid tumors include tumors of hematological origin (hematological cancer), including, e.g., myelomas (e.g., multiple myeloma), leukemias (e.g., Waldenstrom's syndrome, chronic lymphocytic leukemia, other leukemias), and lymphomas (e g, B-cell lymphomas, non-Hodgkins lymphoma). Solid tumors can originate in organs, and include cancers such as brain cancer (e.g. glioblastoma), or other solid tumors such as lung, breast, prostate, ovary, colon, kidney, and liver cancer.

The term “cancer cell” as used herein means a cell that shows aberrant cell growth, such as increased cell growth. A cancerous cell may be a hyperplastic cell, a cell that shows a lack of contact inhibition of growth in vitro, a tumor cell that is incapable of metastasis in vivo, or a metastatic cell that is capable of metastasis in vivo. A cancer cell also includes a cancer stem cell.

The term “cannabinoid” as used herein refers to an agent found in Cannabis sativa or Cannabis indica including tetrahydrocannabinol (THC), tetrahydrocannabinolic acid (THC Acid), cannabidiol (CBD), cannabidiolic acid (CBD Acid), cannabigerolic acid, cannabigerol, cannabigerovarinic acid, cannabigerolovarin, cannabichromenic acid, cannabichromene, cannabidivarin, cannabidivarinic acid, tetrahydrocannabivarinic acid, tetrahydrocannabivarin, cannabivarinic acid, cannabivarin, cannabinolic acid, cannabinol, and isomers thereof, and mixtures of two or more of the foregoing thereof.

The term “cannabinoid containing PEV(s)” or “CPEVs” as used herein refers to PEVs that contain one or more cannabinoids.

The term “co-administration” or “co-administering” as used herein refers to the administration of an active agent before, concurrently, or after the administration of another active agent such that the biological effects of either agents overlap. The combination of agents as taught herein can act synergistically to treat or prevent the various diseases, disorders or conditions described herein. Using this approach, one may be able to achieve therapeutic efficacy with lower dosages of each agent, thus reducing the potential for adverse side effects.

As used herein, the term “concentrating” refers to a process whereby a molecule or structure of interest that is in a mixture that has been subjected to that process has a greater concentration after the process as compared to the concentration of the molecule in the mixture before the process.

As used herein, the term “enriching” (and “enriched” and the like) refers to a process whereby a molecule of interest or structure of interest that is in a mixture has an increased ratio of the amount of that molecule to the amount of other undesired components in that mixture after the enriching process as compared to before the enriching process.

“Excipient(s) for intranasal delivery” are described e.g. in US2013/0337067 and include pharmaceutically acceptable diluents, preservatives, solubilizers, emulsifiers, adjuvants and/or carriers. Such compositions are liquids or lyophilized or otherwise dried formulations and include diluents of various buffer content (e.g., Tris-HCl, acetate, phosphate), pH and ionic strength, additives such as albumin or gelatin to prevent absorption to surfaces, and detergents (e.g. Tween 20™, Tween 80™, Pluronic F68™, bile acid salts). The pharmaceutical composition can comprise pharmaceutically acceptable solubilizing agents (e.g. glycerol, polyethylene glycol), anti-oxidants (e.g. ascorbic acid, sodium metabisulfite), preservatives (e.g. thimerosal, benzyl alcohol, parabens), bulking substances or tonicity modifiers (e.g. lactose, mannitol).

“Exogenous payload” refers to a payload loaded into PEVs that is from an external source, i.e., did not originate in the EVs. PEVs may contain a combination of both endogenous and exogenous payloads.

“Endogenous payload” refers to a payload that is naturally occurring in the PEVs. These would include PEVs that are isolated from genetically engineered and non-genetically engineered plants.

As used herein, the term “formulated for delivery to an animal” refers to a PEV composition that includes a pharmaceutically acceptable carrier. As used herein, a “pharmaceutically acceptable” carrier or excipient is one that is suitable for administration to an animal (e.g., human), e.g., without undue adverse side effects to the animal (e.g., human).

“intranasal delivery” refers to extra- and transcellular transport through the olfactory and respiratory mucosal epithelium from the nasal cavity to the brain. This physiological process is described in detail in Van Woensel et al. (2013), cited above. Devices for intranasal delivery are commercially available and are known under the trade names Vianase (Kurve Technologies, USA) DirectHaler (Denmark) or OptiMist (Norway).

As used herein, the term “isolating,” or “to isolate,” refers to any artificial (i.e., not naturally occurring) process for treating a starting material, where the process results in a more useful form of a molecule or structure of interest (e.g. extracellular vesicles) that is in the starting material. The “more useful form” of the molecule or structure of interest can be characterized in a variety of ways, no one of which is limiting. For example, as used herein, certain embodiments provide methods for isolating extracellular vesicles from a cannabis plant or cells thereof. Further, for example, the process for isolating can result in:
(i) the molecule of interest or structure of interest having a greater concentration in the isolated form compared to the starting material (e.g., concentrating),
(ii) the removal of any amount or any type of impurities from the starting material (e.g., purifying),
(iii) an increase in the ratio of the amount of molecule of interest or structure of interest to the amount of any undesired component in the starting material (e.g., enriching),
(iv) any artificial process for removing a molecule or structure of interest from its natural source or location;
(v) any artificial process for separating a molecule or structure of interest from at least one other component with which it is normally associated (e.g., purifying), or
(vi) any combination of (i), (ii), (iii), (iv) or (v).

Similarly, as used herein, the term “isolated” generally refers to the state of the molecule or structure of interest after the starting material has been subjected to a method for isolating the molecule of interest. That is to say, isolating a molecule of interest or structure of interest from a starting material will produce an isolated molecule. For example, the methods of the invention are used to produce preparations of isolated extracellular vesicles.

The term “neurodegenerative disease” as used herein refers to a condition that involve degeneration of neurons in the CNS system. Examples of neurodegenerative diseases include, but are not limited to, one or more of Alzheimer's disease, Parkinson's disease, Huntington's disease, motor neuron disease, spinocerebellar ataxia, and spinal muscular atrophy.

The term “CNS injuries” as used herein refers to injury or trauma to the CNS of a subject. Examples of CNS injuries include, but are not limited to one or more of stroke, traumatic brain injury, concussion, and spinal cord injury.

The term “inflammatory condition” as used herein refers to refers to any inflammatory disease or disorder known in the art whether of a chronic or acute nature. Examples of inflammatory conditions treatable by the compositions described herein include auto-immune diseases (e.g. multiple sclerosis, encephalitis and the like.

The term “payload” as used with respect to PEVs refers to constituents contained by individual extracellular vesicles. In various embodiments, the extracellular vesicle membrane comprises an interior surface and an exterior surface and encloses an internal space. In certain embodiments, the payload is enclosed within the internal space. In other embodiments, the payload is displayed on the external surface of the extracellular vesicle. In other embodiments, the payload spans the membrane of the extracellular vesicle. In various embodiments, the payload comprises nucleic acids, proteins, carbohydrates, lipids, small molecules, and/or combinations thereof. The PEVs may contain both exogenous and/or endogenous payloads. Further, endogenous payloads can be enriched above that which is found in nature. Payloads for loading into PEVs (i.e. exogenous payloads) may include a select therapeutic agent that are artificially chemical synthesized and/or isolated from other sources, including other plant or fungal material.

The term “PEV composition” refers to a PEVs combined with a carrier.

As used herein, the term “plant” refers to whole plants (e.g., whole seedlings or whole adult plants), plant organs, plant parts, plant tissues, seeds, plant cells, seeds, and progeny of the same.

As used herein, the term “plant extracellular vesicle” or “PEV” refers to a lipid structure (e.g., a lipid bilayer, unilamellar, multilamellar structure; e.g., a vesicular lipid structure), that is about 5-2000 nm (e.g., at least 5-1000 nm, at least 5-500 nm, at least 400-500 nm, at least 25-250 nm, at least 50-150 nm, or at least 70-120 nm) in diameter that is derived from (e.g., enriched, isolated or purified from) a plant source or segment, portion, or extract thereof, including lipid or non-lipid components (e.g., peptides, nucleic acids, or small molecules) associated therewith and that has been enriched, isolated or purified from a plant, a plant part, or a plant cell or from a culture medium in which a plant, plant part, or plant cell has been cultured (e.g., a culture medium of a plant cell culture or a hydroponic culture, e.g., secreted PEVs), the enrichment or isolation removing one or more contaminants or undesired components originating from the source plant, plant part, or plant cell or from the culture medium. In some examples, the isolation comprises removing an intact plant or plant part from the culture medium (e.g., a culture medium of a hydroponic system), e.g., removing the plant or plant part without disrupting (e.g., physically damaging) the plant or plant part. PEVs may be highly purified preparations of naturally occurring EVs. Preferably, at least 1% of contaminants or undesired components from the source plant are removed (e.g., at least 2%, 5%, 10%, 15%, 20%, 25%, 30%, 40%, 45%, 50%, 55%, 60%, 70%, 80%, 90%, 95%, 96%, 98%, 99%, or 100%) of one or more contaminants or undesired components from the source plant, e.g., plant cell wall components; pectin; plant organelles (e.g., mitochondria; plastids such as chloroplasts, leucoplasts or amyloplasts; and nuclei); plant chromatin (e.g., a plant chromosome); or plant molecular aggregates (e.g., protein aggregates, nucleic acids, proteins, protein-nucleic acid aggregates, lipoprotein aggregates, lipido-proteic structures, or sugars). Preferably, a PEV is at least 30% pure (e.g., at least 40% pure, at least 50% pure, at least 60% pure, at least 70% pure, at least 80% pure, at least 90% pure, at least 99% pure, or 100% pure) relative to the one or more contaminants or undesired components from the source plant as measured by weight (w/w), spectral imaging (% transmittance), or conductivity (S/m). PEVs may encompass exosomes or microvesicles. Generally, PEVs comprise a payload that can be delivered to a cell upon association of the PEV with the cell. Exemplified herein are PEVs obtained from plant material of a Cannabis spp plant. As used herein, csPEV refers to a PEV from a Cannabis spp.

CPEVs may optionally include additional agents, such as heterologous functional agents, e.g., therapeutic agents, polynucleotides, polypeptides, or small molecules. The CPEVs can carry or associate with additional agents (e.g., heterologous functional agents) in a variety of ways to enable delivery of the agent to a target plant, e.g., by encapsulation of the agent, incorporation of the agent in the lipid bilayer structure, or association of the agent (e.g., by conjugation) with the surface of the lipid bilayer structure. Exogenous functional agents can be incorporated into the CPEVs either in vivo (e.g., in planta) or in vitro (e.g., in tissue culture, in cell culture, or synthetically incorporated).

As used herein, the terms “purified” or “partially purified” refers to molecules or structures of interest that are removed from either (1) their natural environment, or from (2) a starting material (i.e., they are isolated), and where (a) at least one impurity from the starting material has been removed, or (b) at least one component with which the molecule is naturally associated has been removed. A “purified” or “partially purified” molecule may still contain additional components that may render future use or study of the molecule sub-optimal, difficult or impossible.

As used herein, the term “purifying” or “to purify” a molecule or structure of interest refers to a process for removing at least one impurity or contaminant from a starting material. For example, purifying a molecule of interest from a starting material refers to a process for removing at least one impurity from the starting material to produce a relatively more pure form of the molecule of interest.

In a certain embodiment, a CPEV composition of this disclosure can be administered to a subject who has symptoms of or is diagnosed with a cancer. A composition of this invention can be administered prophylactically, i.e., before development of any symptom or manifestation of the disease, disorder or condition. Typically, in this case the subject will be at risk of developing the condition. Treating also may comprise treating a subject exhibiting symptoms of a certain disease, disorder or condition.

As used herein, the term “subject” refers to an animal being treated with CPEVs as taught herein. The term includes any animal, preferably a mammal, including, but not limited to, farm animals, zoo animals, companion animals, service animals, laboratory or experimental model animals, sport animals. More specific examples include simians, avians, felines, canines, equines, rodents, bovines, porcines, ovines, and caprines. The term specifically includes humans and human patients. In a specific embodiment, subjects pertain to human cancer patients or humans in need of treatment for cancer, including glioblastoma. A suitable subject for the invention can be any animal, preferably a human, that is suspected of having, has been diagnosed as having, or is at risk of developing a disease that can be ameliorated, treated or prevented by administration of one or more CPEVs. Therefore, a “subject in need” refers to a subject as defined herein that is suspected of having, has been diagnosed as having, or is at risk of developing a disease that can be ameliorated, treated or prevented by administration of one or more CPEVs and compositions including same.

As used herein, the term “substantially purified” refers to molecules or structures of interest that are removed from their natural environment or from a starting material (i.e., they are isolated) and where they are largely free from other components with which they are naturally associated or substantially free of other components that may render future use or study sub-optimal, difficult or impossible.

A “therapeutically effective amount” refers to an amount which, when administered in a proper dosing regimen, is sufficient to reduce or ameliorate the severity, duration, or progression of the disorder being treated (e.g., cancer), prevent the advancement of the disorder being treated (e.g., cancer), cause the regression of the disorder being treated (e.g., cancer), or enhance or improve the prophylactic or therapeutic effects(s) of another therapy. The full therapeutic effect does not necessarily occur by administration of one dose and may occur only after administration of a series of doses. Thus, a therapeutically effective amount may be administered in one or more administrations per day for successive days.

The terms “treat”, “treating” or “treatment of” as used herein refers to providing any type of medical management to a subject. Treating includes, but is not limited to, administering a composition to a subject using any known method for purposes such as curing, reversing, alleviating, reducing the severity of, inhibiting the progression of, or reducing the likelihood of a disease, disorder, or condition or one or more symptoms or manifestations of a disease, disorder or condition.

### Plant Extracellular Vesicles

A plant extracellular vesicle including exosomes, are nanoscale membrane-enclosed particles implicated in intercellular communication to facilitate transport of proteins and genetic material. Plant extracellular vesicles can have, but not be limited to, a diameter of greater than about about 5-2000 nm (e.g., at least 5-1000 nm, at least 5-500 nm, at least 400-500 nm, at least nm, at least 50-150 nm, or at least 70-120 nm).

As used herein, plant extracellular vesicles can also include any shed membrane bound particle that is derived from either the plasma membrane or an internal membrane. Plant extracellular vesicles can also include cell-derived structures bounded by a lipid bilayer membrane arising from both herniated evagination (blebbing) separation and sealing of portions of the plasma membrane or from the export of any intracellular membrane-bounded vesicular structure containing various membrane-associated proteins. Plant extracellular vesicles can also include membrane fragments.

Plant extracellular vesicles can be directly assayed from the biological samples, such that the level of plant extracellular vesicles is determined or the one or more biomarkers of the plant extracellular vesicles are determined without prior isolation, purification, or concentration of the plant extracellular vesicles. Alternatively, plant extracellular vesicles may be isolated, purified, or concentrated from a sample prior to analysis.

Analysis of a plant extracellular vesicle can include quantitating the amount one or more plant extracellular vesicle populations of a biological sample. For example, a heterogeneous population of plant extracellular vesicles can be quantitated, or a homogeneous population of plant extracellular vesicles, such as a population of plant extracellular vesicles with a particular biomarker profile, a particular bio-signature, or derived from a particular cell type (cell-of-origin specific plant extracellular vesicles) can be isolated from a heterogeneous population of plant extracellular vesicles and quantitated. Analysis of a plant extracellular vesicle can also include detecting, quantitatively or qualitatively, a particular biomarker profile or a bio-signature, of a plant extracellular vesicle, as described below.

A plant extracellular vesicle can be stored and archived, such as in a bio-fluid bank and retrieved for analysis as necessary. A plant extracellular vesicle may also be isolated from a biological sample that has been previously harvested and stored from a living or deceased subject. In addition, a plant extracellular vesicle may be isolated from a biological sample or isolated from an archived or stored sample. Alternatively, a plant extracellular vesicle may be isolated from a biological sample and analyzed without storing or archiving of the sample. Furthermore, a third party may obtain or store the biological sample, or obtain or store the plant extracellular vesicles for analysis.

An enriched population of plant extracellular vesicles can be obtained from a biological sample. For example, plant extracellular vesicles may be concentrated or isolated from a biological sample using size exclusion chromatography, density gradient centrifugation, differential centrifugation, nanomembrane ultrafiltration, immunoabsorbent capture, affinity purification, microfluidic separation, or combinations thereof. These methods can be used to separate plant extracellular vesicles from contaminants.

Size exclusion chromatography, such as gel permeation columns, centrifugation or density gradient centrifugation, and filtration methods can be used. For example, plant extracellular vesicles can be isolated by differential centrifugation, anion exchange and/or gel permeation chromatography (for example, as described in U.S. Pat. Nos. 6,899,863 and 6,812,023), sucrose density gradients, organelle electrophoresis (for example, as described in U.S. Pat. No. 7,198,923), magnetic activated cell sorting (MACS), or with a nanomembrane ultrafiltration concentrator. Various combinations of isolation or concentration methods can be used.

Isolation or enrichment of plant extracellular vesicles from biological samples can also be enhanced by use of sonication (for example, by applying ultrasound), or the use of detergents, other membrane-active agents, or any combination thereof.

### Administration

In some embodiments, the composition embodiments comprising CPEVs described herein will be administered intranasally to a mammalian subject in need thereof using a level of pharmaceutical composition that is sufficient to provide the desired physiological effect. The mammalian subject may be a domestic animal or pet but preferably is a human subject. The level of pharmaceutical composition needed to give the desired physiological result is readily determined by one of ordinary skill in the art. Other parameters that may be taken into account in determining dosage for the pharmaceutical composition embodiments described herein may include disease state of the subject or age of the subject. The composition will typically include an excipient for intranasal delivery.

The compositions may take the form of suspensions, solutions or emulsions in oily or aqueous vehicles and may contain formulatory agents such as suspending, stabilizing and/or dispersing agents. In some embodiments, the composition embodiments described herein may be administered orally or intravenously (e.g. via parenteral nutritional therapy) to a subject via an emulsion. The emulsion may include, in some embodiments, an aqueous continuous phase and a dispersed phase. The boundary between the phases called the “interface”. The present emulsions are adapted for application to a mucosal surface of a vertebrate animal, preferably a mammal, including humans. These compositions improve the permeability and bioavailability of active compounds after application to a mucous surface. Mucosal surfaces of interest include the intestinal mucosa. Use of bioadhesive polymers in pharmaceutical emulsions affords enhanced delivery of drugs in bioadhesive polymer-coated suspensions, in some examples. Bioadhesive pharmaceutical emulsions may be used to deliver the described herein to: a) prolong the residence time in situ, thereby decreasing the number of drug administrations required per day; and b) may be localized in the specified region to improve and enhance targeting and bioavailability of delivered drugs.

The ability to retain and localize a CPEV delivery emulsion in a selected region leads to improved bioavailability, especially for drugs exhibiting a narrow window of adsorption due to rapid metabolic turnover or quick excretion. Intimate contact with the target absorption membrane improves both the extent and rate of drug absorption.

Bioadhesion is the characteristic of certain natural and synthetic polymers of binding to various biological tissues. Of particular interest are polymers which bind to the mucous lining that covers the surface of many tissues which communicate directly or indirectly with the external environment, such as the nasal mucosa, for example. Mucus binding polymers may be referred to as mucoadhesive. Several bioadhesive, and specifically mucoadhesive, polymers are known. The chemical properties of the main mucoadhesive polymers are summarized as follows:
a. strong H-bonding groups (—OH, —COOH) in relatively high concentration;
b. strong anionic charges;
c. sufficient flexibility of polymer backbone to penetrate the mucus network or tissue crevices;
d. surface tension characteristics suitable for wetting mucus and mucosal tissue surfaces; and
e. high molecular weight.

Bioadhesive polymers may be used in the pharmaceutical composition embodiments described herein, examples of bioadhesive polymers currently used in pharmaceutical preparations include: carboxymethylcellulose (CMC), hydroxypropylmethylcellulose (HPMC), polyacrylic and polymethacrylic acid and their derivatives, pectin, alginic acid, chitosan, polyvinylpyrrolidone, hyaluronic acid, and polyvinyl alcohol. The most frequently used polymer is Carbopol (Carbomer), which is a high molecular weight polyacrylic acid polymer. It is used in many formulations for bioadhesive drug delivery systems, as a suspending agent, as a tablet coating, and in ocular suspensions.

Pharmaceutical composition embodiments described herein may include the composition comprising PEVs, CPEVs or csPEVs incorporated into inert lipid carriers such as oils, surfactant dispersions, emulsions, liposomes etc. Self-emulsifying formulations are ideally isotropic mixtures of oils, surfactants and co-solvents that emulsify to form fine oil in water emulsions when introduced in aqueous media. Fine oil droplets would pass rapidly from stomach and promote wide distribution of drug throughout the GI tract, thereby overcome the slow dissolution step typically observed with solid dosage forms. These embodiments may provide control release self-emulsifying pellets, microspheres, tablets, capsules etc. that increase the use of “self-emulsification.”

Intranasal delivery is the typical mode of administration to deliver the PEVs, CPEVs or csPEVs to a subject in need. However, in alternative embodiments, other methods of administration are contemplated. Accordingly, suitable methods for administering a PEV, CPEV or csPEV containing composition in accordance with the methods of the presently-disclosed subject matter include, but are not limited to, oral administration, systemic administration, parenteral administration (including intravascular, intramuscular, and/or intraarterial administration), oral delivery, buccal delivery, rectal delivery, subcutaneous administration, intraperitoneal administration, inhalation, dermally (e.g., topical application), intratracheal installation, surgical implantation, transdermal delivery, local injection, and hyper-velocity injection/bombardment. Where applicable, continuous infusion can enhance drug accumulation at a target site (see, e.g., U.S. Pat. No. 6,180,082). In some embodiments of the therapeutic methods described herein, the therapeutic compositions are administered orally, intravenously, intranasally, or intraperitoneally to thereby treat a disease or disorder.

Regardless of the route of administration, the compositions of the presently-disclosed subject matter typically not only include an effective amount of a PEVs, but are typically administered in amount effective to achieve the desired response. As such, the term “effective amount” is used herein to refer to an amount of the therapeutic composition (e.g., a PEVs and a pharmaceutically vehicle, carrier, or excipient) sufficient to produce a measurable biological response (e.g., reduction in cancer cells). Actual dosage levels of active ingredients in a therapeutic composition of the present invention can be varied so as to administer an amount of the active compound(s) that is effective to achieve the desired therapeutic response for a particular subject and/or application. Of course, the effective amount in any particular case will depend upon a variety of factors including the activity of the therapeutic composition, formulation, the route of administration, combination with other drugs or treatments, severity of the condition being treated, and the physical condition and prior medical history of the subject being treated. Preferably, a minimal dose is administered, and the dose is escalated in the absence of dose-limiting toxicity to a minimally effective amount. Determination and adjustment of a therapeutically effective dose, as well as evaluation of when and how to make such adjustments, are known to those of ordinary skill in the art. Background information on formulations of extracellular vesicles and loading EVs with exogenous payloads is taught in U.S. Pat. No. 10,723,782, and Tran et al. “Exosomes as Nanocarriers for Immunotherapy of Cancer and Inflammatory Diseases. (2015) Clin Immunol. PMID: 25842185.

### Kits

In view of the new findings described herein that cannabinoids packaged in plant extracellular vesicles can improve delivery of cannabinoids to tissues or cells (tumor or normal cells), kits are provided that include a container for housing the PEVs and an applicator for delivery of the PEVs. In a specific example, the applicator includes a tapered spout for insertion into a nostril of a subject that attaches to the container. The CPEV compositions described herein can be administered to the nasal cavity in any suitable form, for example in the form of drops or sprays.

Methods suitable for administering a CPEV or csPEV composition to the nasal cavity will be well known by the person of ordinary skill in the art. Any suitable method may be used. The preferred method of administration is the use of a spray device. Spray devices can be single (unit) dose or multiple dose systems, for example comprising a bottle, pump and actuator, and are available from various commercial sources including Pfeiffer, Valois, Bespak and Becton-Dickinson. Electrostatic spray devices, such as described in U.S. Pat. No. 5,655,517, are also suitable for the intranasal administration of the compositions of the present invention.

For a spray device, the typical volume of liquid that is dispensed in a single spray actuation is in the range of from 0.01 to 0.15 ml. A typical dosing regimen for a nasal spray product would be in the range of one spray into a single nostril to two sprays into each nostril.

The present invention also provides a spray device loaded with a composition as defined above.

### EXAMPLES

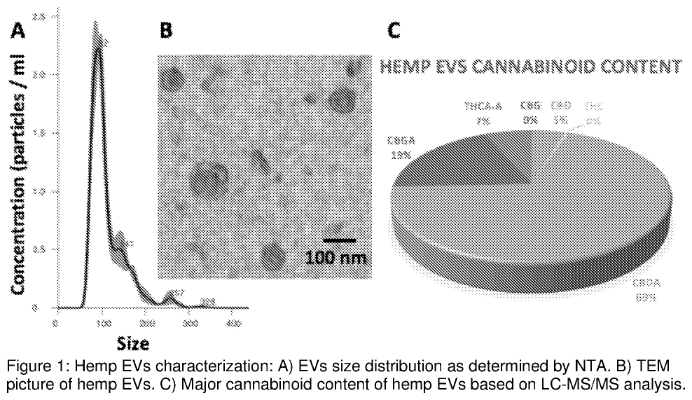

To this end, we have discovered and isolated naturally occurring hemp extracellular vesicles (EVs) that are enriched with a payload of cannabinoids in acidic forms. Floral parts of fresh hemp plant were juiced. Hemp juice was first centrifuged at different speeds (from 2000-35000×g) and filtered through different size paper filters to remove debris and particles larger than 0.221M and then was transferred to ultracentrifuge tubes and centrifuged at 150,000×g for 1 hour in a Beckman Coulter Optima™ XE ultracentrifuge with a Type 45 Ti rotor. The EV pellet was resuspended in sterile PBS and stored in −80° C. freezer. Nanotracking analysis (NTA, Nanosight NS300 instrument) has shown that hemp EVs have a median diameter of 112.6 nm (FIG. 1A). Transmission electron microscopy (TEM, Tecnai G2 F20-TWIN) evaluation has validated the size and morphology of hemp EVs with a lipid bilayer structure typical of the EVs (FIG. 1B). Major cannabinoid content of hemp EVs (CBD, CBD-A, THC, THC-A, CBG, CBG-A) was quantified using Liquid chromatography tandem mass spectrometry (LC-MS/MS). Analytes were extracted from the EVs using solid phase extraction (C18 cartridge) method. EV samples were spiked with deuterated internal standards prior to the extraction to check recovery and correction of extraction efficiency. Our results have shown that CBD-A, CBG-A, THC-A, CBD, and THC represent 69.1±2.1%, 19.1±1.6%, 6.5±0.54%, 4.75±0.26%, and 0.5±0.3% of the total cannabinoids in hemp EVs (FIG. 1C) without any detectable level of CBG.

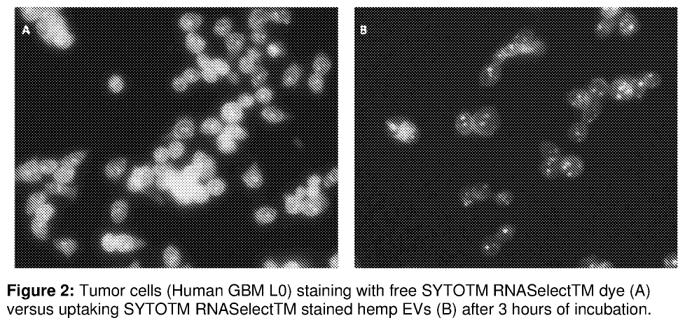

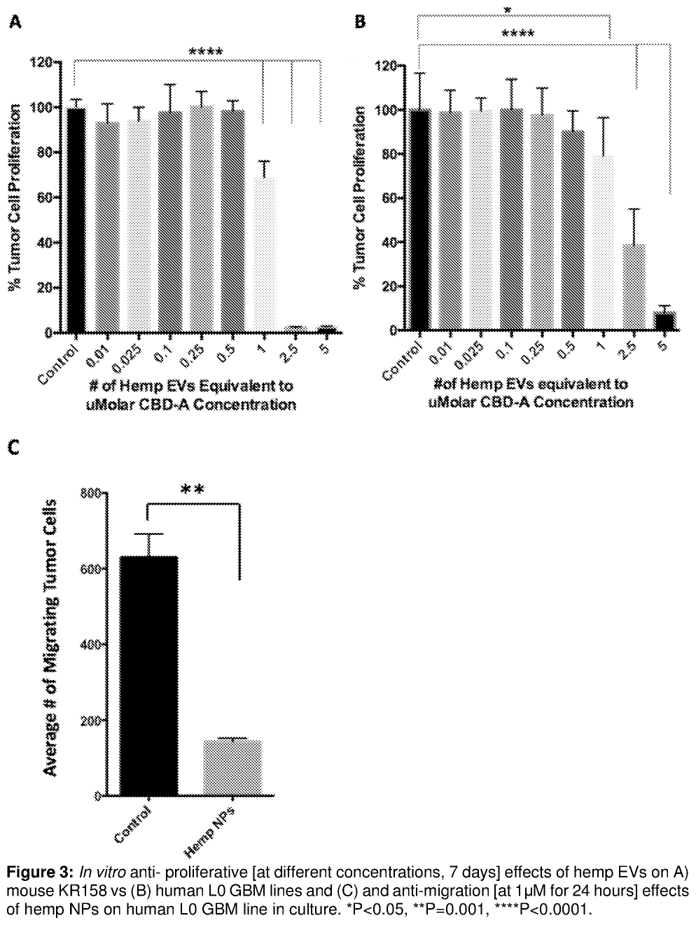

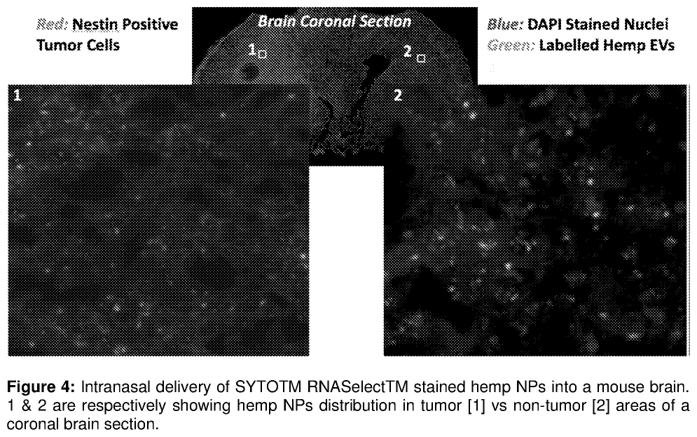

To assay the uptake of EVs by cells, we labelled hemp EVs with SYTO™ RNASelect™ green fluorescent cell stain as per manufacturer's instructions and removed excess unincorporated dye from the labelled EVs with exosome spin columns (MW 3000). Human glioblastoma tumor cells were plated in laminin coated coverslip in growth medium overnight and then the labeled EVs were added to each well and EVs uptake by the cells was assessed 3 hours later. Our results show that EVs can be taken up by GBM cells in vitro presenting a characteristic dotted staining pattern in contrast to a uniform cytoplasmic staining pattern of cells exposed to the free dye (FIG. 2). In vitro anti-glioma effects of the hemp EVs were studied both in human and murine GBM cell lines. First, the mean CBD-A concentration per EV and then the number of EVs equal to a certain CBD-A concentration was calculated based on the LC-MS/MS results. Tumor cells were plated in 96 wells in neurosphere growth medium supplemented with hemp EVs corresponding to different CBD-A concentrations. After 7 days, the mean fluorescent intensity (as an index of cell proliferation) was measured and normalized based on that of the control condition using Alamar Blue assay. Hemp EVs have shown significant anti-glioma effect starting at a number that is equivalent to or more than 1 μM of CBD-A (FIG. 3A,B). Overnight incubation of human GBM tumor cells with a non-killing dose of hemp EVs effectively reduces GBM cell migration in a transwell migration assay (FIG. 3C). In order to test the intranasal delivery of hemp EVs, we used a C57/B16 mouse with an established KR-158 tumor in the left hemisphere and delivered 12μl of SYTO™ RNASelect™ green fluorescent labelled hemp EVs in both nostrils (2 μl/nostril every 5 minutes).

The animal were sacrificed 24 hours later and brain tissue was fixed, cryosectioned and stained for anti-nestin antibody and counterstained with DAPI to distinguish the tumor vs non-tumor areas of the brain. labelled hemp EVs are scattered throughout the brain hemispheres (FIG. 4).

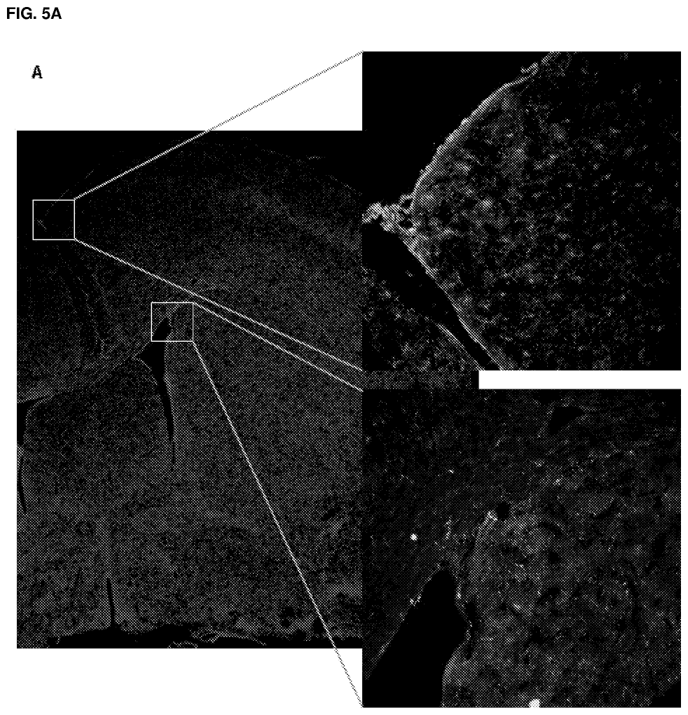

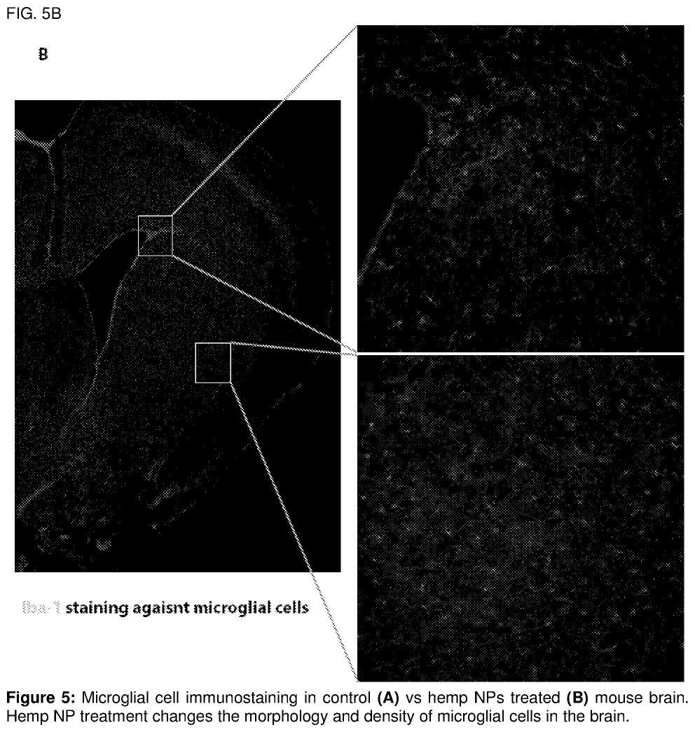

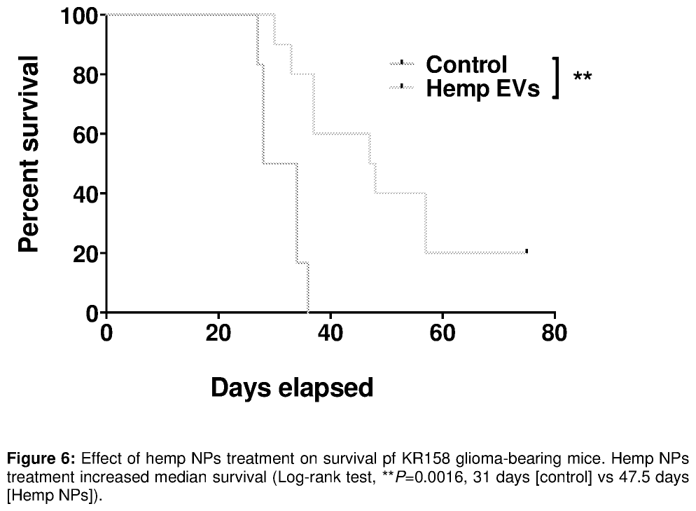

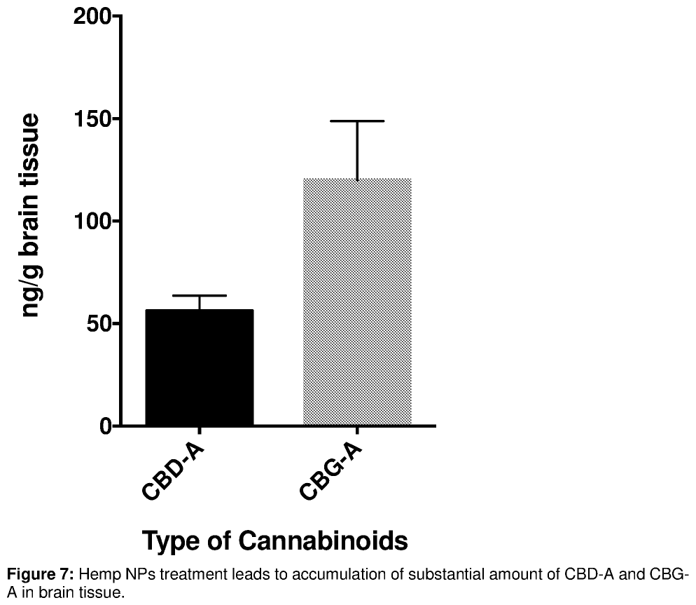

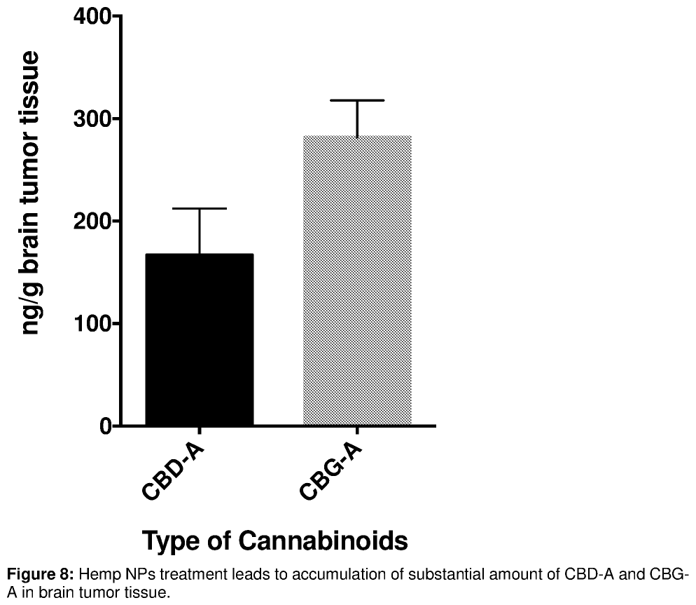

To assay antitumor effect of hemp EVs, a cohort of animals were implanted with KR-158 tumor. Three days after implantation, the animals were randomly placed in two groups receiving either PBS (vehicle of hemp EVs, control group) or hemp EVs every day intranasally for 35 days (twice daily with 6 hour interval between the two intranasal deliveries). Immunofluorescence analysis of brain tissue for Iba-1 in control and hemp EV treated group showed a distinct difference in microglial cell morphology and density between the two groups (FIG. 5A,B). Animal survival analysis using Log-rank test revealed that hemp EV therapy significantly increased animal survival and slowed down tumor growth (FIG. 6). LC-MS/MS analysis of cannabinoid content of the brain (FIG. 7) and KR-158 tumor tissue (FIG. 8) showed that intranasal hemp EV delivery leads to a substantial amount of CBD-A and CBG-A in these tissues. This discovery is exciting because delivering hemp EVs with its natural cannabinoid payload directly into the brain via noninvasive intranasal approach will improve killing of malignant brain tumor cells, thereby increasing treatment response, improving patient survival and potentially achieving a cure. Intranasal delivery of cannabinoids in natural nanovesicle form holds a great promise circumventing the systemic effects of cannabinoid oral delivery and bypassing first pass metabolism in liver. Additionally, intranasal delivery method of cannabinoids represents a promising, safe and gentle alternative to the invasive, inconvenient and costly delivery methods such as intratumoral approach delivery approach for brain tumors. Hemp EVs have an endogenous payload of cannabinoids and the technology can rapidly move into clinical testing.

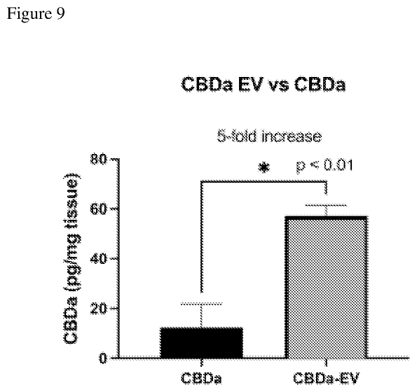

Cannabidiolic acid (CBDa) was dissolved in PBS (64 ng/10 ul) or hemp EVs, isolated, and diluted to have the same CBDa concentration as purified CBDa. C57/B6 mice received a single dose (10 μl purified CBDa or 10111 of hemp EVs) intranasally. One hour after treatment animals were killed, brains removed and CBDa content analyzed with Linear Ion Trap Quadrupole LC-MS/MS (AB SCIEX Instruments). CBDa levels in the brain were 5-fold higher when delivered with hemp EVs (FIG. 9).

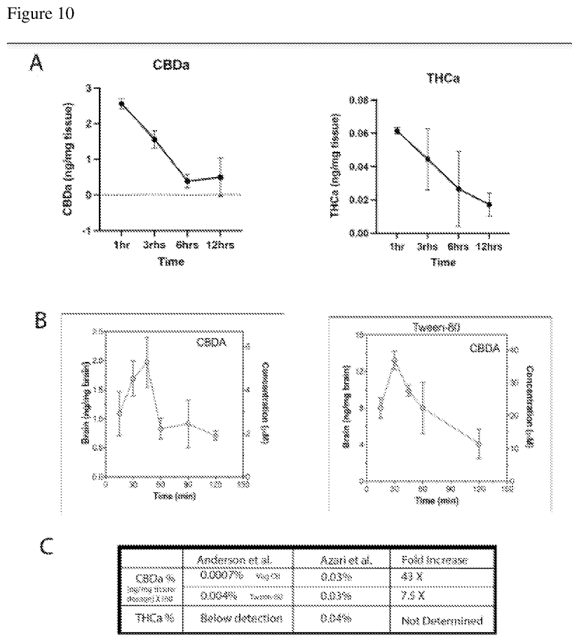

C57/B6 mice were gavaged with hemp derived EV containing approximately 6.4 μg of CBDa and 0.140 μg THCa. One, 3, 6 and 12 hours post gavage animals were killed, brains removed and CBDa and THCa content analyzed with Linear Ion Trap Quadrupole LC-MS/MS [AB SCIEX Instruments]. Peak levels of CBDa and THCa were seen at 1 hour post gavage, with concentrations being 2.5 ng/mg and 0.06 ng/mg of brain tissue, respectively (FIG. 10A). This can be compared to Anderson et al., 2019* who delivered by IP injection approximately 300 μg of CBDa or THCa to C57/B6 mice, either dissolved in vegetable oil or in ethanol Tween-80 solution and analyzed plasma and brain levels of each compound (FIG. 10B). While THCa levels were below detection in the brain, CBDa demonstrated a Cmax at 30 mins with concentrations of 2.0 ng/mg and 13.2 ng/mg in vegetable oil or Tween-80, respectively. Comparison between Anderson et al., 2019 and plant EV delivery (FIG. 10C). Percentage of CBDa or THCa in brain (ng/mg of tissue/dosage) for each method demonstrates the plant EV have a 7.5-43 fold increase in CBDa brain penetration over Tween-80 or vegetable oil vehicles, respectively. THCa penetration with plant EVs were similar to CBDa.

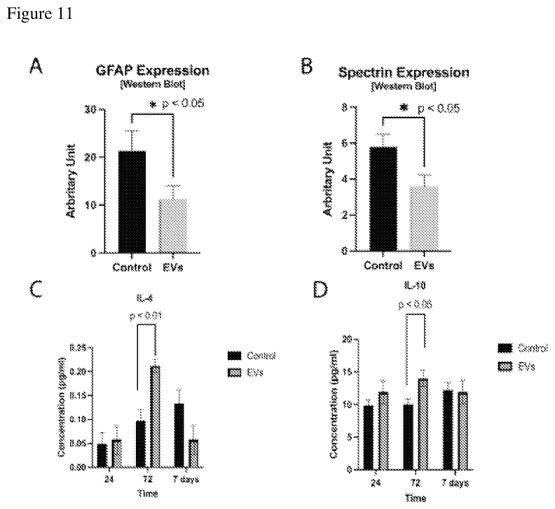

TBI was induced using a unilateral fluid percussion injury (FPI) model and the animals were treated with either 10 μl of PBS or hemp EVs one hour after injury, twice daily for 7 days. Animals were bled at 24 hrs, 72 hrs and 7 days post-TBI and sacrificed on day 7. Western blot analysis of GFAP and Spectrin protein levels in the injured cortex of the control and plant EV treated TBI animals (FIG. 11A-B). A significant reduction in GFAP and Spectrin protein levels are seen in the plant EV treated TBI animals. Inflammatory cytokine analysis using (V-PLEX proinflammatory panel (Mesoscale Diagnostics)) at 24 hours, 72 hours and 7 days post injury reveal that plant EVs result in a significant increase in anti-inflammatory cytokines at 72 hours for IL-4 (FIG. 11C) & IL-10 (FIG. 11D).

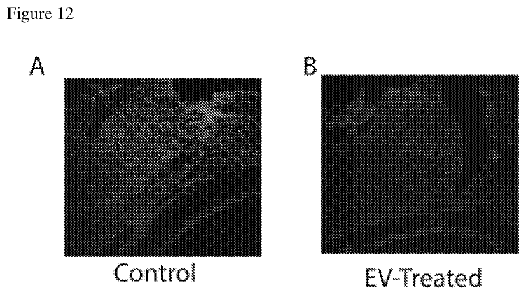

TBI was induced using a unilateral fluid percussion injury (FPI) model and the animals were treated with either 10 μl of PBS or hemp EVs one hour after injury, twice daily for 7 days. Animals were sacrificed on day 7 for brain histological analysis. Control brains IBA-11 (rabbit anti-IBA-1, Encor biotechnology) is upregulated in microglia signifying their activation (FIG. 12A). EV treated animals demonstrate a qualitative reduction in IBA-1 microglia expression supporting the notion that plant EVs are able to attenuate inflammation and microglia activation following TBI (FIG. 12B).

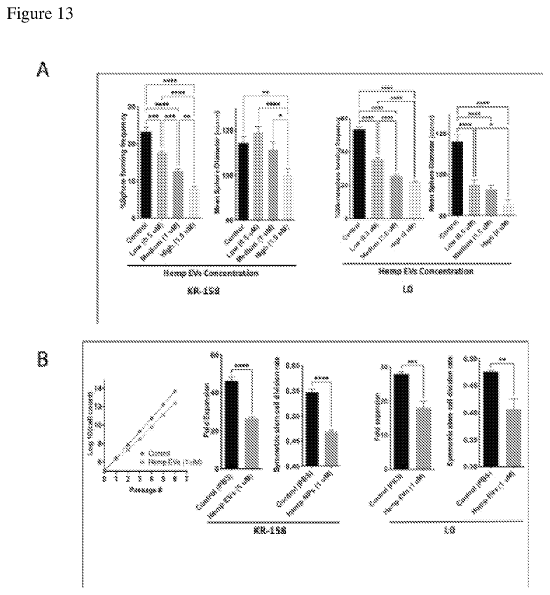

Plant EV increased survival of KR-186luc glioma bearing mice. EV treatment increased median survival (log-rank test, from 31 to 42 days) (FIG. 13).

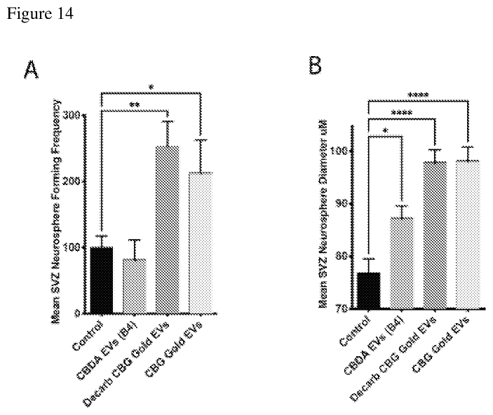

After performing a dose-response analysis to determine the effective antiproliferative concentration (LD50) of the hemp EVs, glioma (mouse KR-158 and human LO) cells were plated in the neurosphere assay culture and treated with three doses of hemp EVs (number of EVs corresponding to their CBDa concentration) that were less than LDS50. After 7 days in culture the number and size of neurospheres were determined in each condition. With increasing concentration of the hemp EVs, both the neurosphere forming frequency and neurosphere size decrease which together shows antiproliferative effect of the hemp EVs on glioma cells (FIG. 14A). Using a mathematical model, serial passaging of the glioma cells in neurophere culture supplemented with an effective antiproliferative dose of the hemp EVs (1 μM) have shown that hemp EVs significantly reduce symmetric cell division in glioma stem cells resulting in a significant decrease in glioma tumor cells expansion over time (FIG. 14B).

Different hemp cultivars (B4 CBDa and Gold CBGa enriched) were used to isolated EVs, which were subsequently concentrated and delivered intranasally twice a day for 7 days. Animals were killed, subventricular zone micro-dissected, tissue dissociated and placed in the Neurosphere Assay. Seven to 10 days later the number of spheres and their diameter where quantitated. Sphere-forming frequency is a reflection of the number of NSCs in vivo and was increased by the Gold EVs (FIG. 15A). B4 cultivar, which is enriched in CBDa did not increase the number of NSCs while both Gold EVs, containing decarboxylated CBG and the acidic form of CBG (CBGa), significantly increased the number of NSCs in the brain. Neurosphere diameter is a metric for proliferation of neural progenitor cells (which are unique and distinct from NSCs) (FIG. 15B). Both the B4 and Gold cultivars increased the proliferation of neural progenitors. Together, these data demonstrate the ability of plant-derived EV to deliver therapeutic drugs to the brain via intranasal delivery.

Given the ability to both bioengineer the hemp plant for enriched production of certain cannabinoids (or harvest EVs from different hemp cultivars) provides a cannabinoid delivery system that can be used to treat non-cancer conditions such as concussion, injury and degenerative diseases. Commercial relevance for this technology is high given several ongoing clinical trials evaluating cannabinoids in brain cancers, neurodegenerative disease, pain, etc.

Although the present invention has been described in considerable detail with reference to certain preferred versions thereof, other versions are possible. Therefore, the spirit and scope of the appended claims should not be limited to the description of the preferred versions contained herein.

The reader's attention is directed to all papers and documents which are filed concurrently with this specification and which are open to public inspection with this specification, and the contents of all such papers and documents are incorporated herein by reference.

All the features disclosed in this specification (including any accompanying claims, abstract, and drawings) may be replaced by alternative features serving the same, equivalent or similar purpose, unless expressly stated otherwise. Thus, unless expressly stated otherwise, each feature disclosed is one example only of a generic series of equivalent or similar features. Any element in a claim that does not explicitly state “means for” performing a specified function, or “step for” performing a specific function, is not to be interpreted as a “means” or “step” clause as specified in 35 U.S.C § 112, sixth paragraph. In particular, the use of “step of” in the claims herein is not intended to invoke the provisions of 35 U.S.C § 112, sixth paragraph.

What is claimed is:

1. A method for treating a brain cancer in a subject in need, comprising administering to the subject in need a therapeutically effective amount of cannabinoid containing plant extracellular vesicles (CPEVs).

2. The method of claim 1, wherein the composition is administered by inhalation, intranasally, orally, intravenously, subcutaneously or intramuscularly.

3. The method of claim 1 or 2, wherein the CPEVs are administered intranasally.

4. The method of any of claims 1-3, wherein the administering comprises administering the CPEVs intranasally using a nasal delivery device selected from the group consisting of an intranasal inhaler, an intranasal spray device, an atomizer, a nebulizer, a metered dose inhaler (MDI), a pressurized dose inhaler, an insufflator, a unit dose container, a pump, a dropper, a nasal spray bottle, a squeeze bottle and a bi-directional device.

5. The method of any of claims 1-4, further comprising co-administering an adjunct cancer therapy protocol.

6. The method of claim 5, wherein the adjunct cancer therapy protocol comprising treating the mammal with radiation before, during, or after the administration of the CPEVs

7. The method of claim 5, wherein the adjunct cancer therapy protocol comprising an additional chemotherapeutic agent.

8. A composition comprising CPEVs and a pharmaceutically acceptable carrier.

9. The composition of claim 8, wherein the pharmaceutically acceptable carrier comprises an excipient for intranasal delivery.

10. A device comprising a container, wherein a CPEV composition is disposed within said container, and a spout associated with the container that is configured for insertion into a nostril of a subject in need.

11. A method for treating a neurodegenerative disease in a subject in need, comprising administering to the subject in need a therapeutically effective amount of cannabinoid containing plant extracellular vesicles (CPEVs).

12. The method of claim 11, wherein the composition is administered by inhalation, intranasally, orally, intravenously, subcutaneously or intramuscularly.

13. The method of claim 11, wherein the neurodegenerative disease comprises stroke, Alzheimer's disease, ALS, MS, Parkinson's disease, traumatic brain injury, or aging.

14. A method for treating a CNS injury in a subject in need, comprising administering to the subject in need a therapeutically effective amount of cannabinoid containing plant extracellular vesicles (CPEVs).

15. The method of claim 14, wherein the composition is administered by inhalation, intranasally, orally, intravenously, subcutaneously or intramuscularly.

16. A method for treating an inflammatory condition in a subject in need, comprising administering to the subject in need a therapeutically effective amount of cannabinoid containing plant extracellular vesicles (CPEVs).

17. The method of claim 16, wherein the composition is administered by inhalation, intranasally, orally, intravenously, subcutaneously or intramuscularly.

18. A method for augmenting function of a CNS in a subject, comprising administering to the subject in need a therapeutically effective amount of cannabinoid containing plant extracellular vesicles (CPEVs).

19. The method of claim 18, wherein the composition is administered by inhalation, intranasally, orally, intravenously, subcutaneously or intramuscularly.

20. A method of making csPEVs comprising
obtaining PEVs from a Cannabis spp.; and
loading the PEVs with an exogenous payload.

21. A composition comprising csPEVs loaded with an exogenous payload.

22. A method of delivering a payload to a subject comprising administering an effective amount of the composition of claim 21 to the subject.

23. The method of claim 22, wherein administering comprises intranasal delivery or parenteral delivery of the composition.

24. The method of claim 23, wherein the composition is administered by inhalation, intranasally, orally, intravenously, subcutaneously or intramuscularly.

25. A device comprising a container, wherein an amount of the composition of claim 21 is disposed within said container.

26. The device of claim 25, further comprising a spout associated with the container that is configured for insertion into a nostril of a subject in need.

27. The method of any of claims 1-7, wherein the CPEVs contain CBD-A, CBG-A, THC-A, CBD, and THC at 69.1±2.1%, 19.1±1.6%, 6.5±0.54%, 4.75±0.26%, and 0.5±0.3%, of total cannabinoids, respectively.

28. A composition of claim 8, wherein the CPEVs contain CBD-A, CBG-A, THC-A, CBD, and THC at 69.1±2.1%, 19.1±1.6%, 6.5±0.54%, 4.75±0.26%, and 0.5±0.3%, of total cannabinoids, respectively.

29. The method of any of claims 11-19, wherein the CPEVs contain CBD-A, CBG-A, THC-A, CBD, and THC at 69.1±2.1%, 19.1±1.6%, 6.5±0.54%, 4.75±0.26%, and 0.5±0.3%, of total cannabinoids, respectively.
